(19) United States
(12) Patent Application Publication
(10) Pub. No.: US 2022/0204644 A1
(43) Pub. Date: Jun. 30, 2022

(54) COMBINATION OF HER2 ANTIBODIES

(71) Applicant: MAB DISCOVERY GMBH, Polling (DE)

(72) Inventor: Stephan Fischer, Polling (DE)

(21) Appl. No.: 17/606,523

(22) PCT Filed: Apr. 29, 2020
(86) PCT No.: PCT/EP2020/061850

§ 371 Date: Oct. 26, 2021

(30) Foreign Application Priority Data
May 2, 2019 (EP) 19172329.5

(51) Int. Cl. C07K 16/32; C07K 16/28; A61K 45/06

(52) U.S. Cl. CPC C07K 16/32; A61K 2039/507; A61K 45/06; C07K 16/2863

(57) ABSTRACT

The present invention is directed to HER2 antibodies directed against an epitope between amino acids 342-652 of human HER2 for use in the treatment of HER2 related disorders in combination with a second HER2 inhibitor. More specifically the invention relates to methods and uses of MAB270 or antibodies having the same CDRs as MAB270 in combination with trastuzumab or pertuzumab in HER2 positive cancer.

### FIELD OF INVENTION

The present invention is directed to HER2 antibodies directed against an epitope between amino acids 342-652 of human HER2 for use in the treatment of HER2 related disorders in combination with one or more other agents. More specifically the invention relates to methods and uses of MAB270 or antibodies having the same CDRs as MAB270 in combination with trastuzumab or pertuzumab in HER2 positive cancer.

### BACKGROUND

Receptor tyrosine-protein kinase erbB-2, also known as proto-oncogene Neu, Erbb2 (rodent), or ERBB2 (human) is a protein that in humans is encoded by the ERBB2 gene, which is also frequently called HER2 (from human epidermal growth factor receptor 2) or HER2/neu.

HER2 is a member of the human epidermal growth factor receptor (HER/EGFR/ERBB) family. HER2, a known proto-oncogene, is located at the long arm of human chromosome 17 (17q12). Amplification or overexpression of this oncogene has been shown to play an important role in the development and progression of certain aggressive types of cancer.

Accordingly in recent years, the protein has become an important biomarker and target of therapy for many cancer types including breast cancer.

The ErbB family consists of four plasma membrane-bound receptor tyrosine kinases. One of which is EGFR, and the other members being epidermal growth factor receptor, HER3 (neuregulin binding; lacks kinase domain), and HER4. All four contain an extracellular ligand binding domain, a transmembrane domain, and an intracellular domain that can interact with a multitude of signaling molecules and exhibit both ligand-dependent and ligand-independent activity. HER2 can heterodimerise with any of the other three receptors and is considered to be the preferred dimerisation partner of the other ErbB receptors.

Dimerisation results in the autophosphorylation of tyrosine residues within the cytoplasmic domain of the receptors and initiates a variety of signaling pathways. These include the mitogen-activated protein kinase (MAPK) pathway, the phosphoinositide 3-kinase (PI3K/Akt) pathway, phospholipase C y-/protein kinase C (PKC)-, and the Signal transducer and activator of transcription (STAT) pathways. Therefore, signaling through the ErbB family of receptors promotes cell proliferation, differentiation and survival, and consequently must be tightly regulated to prevent uncontrolled cell growth from occurring.

Amplification or overexpression of the HER2 gene occurs in approximately 15-30% of breast cancers. It is strongly associated with increased disease recurrence and poor prognosis. Overexpression is also known to occur in ovarian, stomach, bladder, lung, head and neck and aggressive forms of uterine cancer, such as uterine serous endometrial carcinoma. For example, HER2 is overexpressed in approximately 7-34% of patients with gastric cancer and in 30% of salivary duct carcinomas.

Diverse structural alterations have been identified that cause ligand-independent firing of this receptor, even in the absence of receptor overexpression. For example a substitution of a valine for a glutamic acid in the transmembrane domain can result in the constitutive dimerization of this protein in the absence of a ligand. HER2 mutations have also been found in non-small-cell lung cancers (NSCLC).

Because of its central role in tumor development different strategies for targeting HER2 have been employed in the clinic: 1.) antibodies directed against the extracellular domain of the receptor and 2.) small molecule inhibitors acting on the intracellular kinase domain.

Trastuzumab, a humanized monoclonal antibody directed against the extracellular juxtamembrane domain IV of HER2, leads to decreased HER2 signaling and cell growth inhibition by several proposed mechanisms: (I) decreased ligand independent signaling; (II) increased destruction of HER2 after endocytosis; (Ill) immune activation; and (IV) inhibition of shedding of the extracellular domain. Trastuzumab has been proposed to induce antibody-dependent cellular cytotoxicity (ADCC) as its IgG1 Fc heavy chain domain can bind and activate the Fc receptor of immune effector cells. Consistent with this hypothesis, trastuzumab had decreased anti-tumor activity in mice with deletion of FcγRs, whereas augmenting the response of natural killer cells enhanced antitumor activity.

Subsequent to the approval of trastuzumab, the oral small molecule tyrosine kinase inhibitor lapatinib became available for patients with HER2-positive metastatic breast cancer whose disease had progressed on prior treatment with trastuzumab, taxane and an anthracycline. In contrast to trastuzumab, lapatinib binds to the intracellular adenosine triphosphate binding domain of HER1 and HER2. Accordingly clinical synergy has been demonstrated with trastuzumab and lapatinib combination therapy in patients whose disease had progressed on trastuzumab.

Pertuzumab is a recombinant, humanized monoclonal antibody targeting HER2. Unlike trastuzumab which binds HER2 at juxtamembrane domain IV, pertuzumab binds HER2 at the extracellular dimerization subdomain II critical for homo- and heterodimerization. In this way, pertuzumab blocks HER2 receptor dimerization with Her2 or other HER family members, including EGFR, HER1, HER3, and HER4.

Despite overall promising activity in HER2-positive breast cancer, only half of patients had a tumor response and 50% of patients had progression of their disease by within 1 year, indicating that de novo and acquired resistance to HER2-targeted therapy exists. In general, trastuzumab-resistant tumors show continuing HER2 amplification, high HER2 protein level and dependence on HER2 signaling.

Many mechanisms for resistance to anti-HER2 therapy have been suggested. For example primary resistance is mainly a lack of positive response to therapy and might come through redundancy, inactive target receptor (like truncated HER2 receptors lacking extracellular trastuzumab-binding domain), alternative dimerization patterns within the HER receptor family or incomplete inhibition of HER2 signaling pathways. Also acquired resistance has been reported that is caused by the ability to reactivate pathway signaling at or downstream of the receptor layer such as with activating HER, intrinsic alterations of HER2 or loss of downstream pathway negative-regulating mechanisms. Other described mechanisms are the upregulation of other tyrosine-kinase-receptors or crosstalk between estrogen-receptors and HER2 pathways.

Thus, there is a need for the development of novel, more effective antibodies and methods of treatment that can be used in follow-up therapies when results of the gold-standard therapy with trastuzumab (and chemotherapy) are not satisfying or as an alternative in combination with existing antibodies.

### DESCRIPTION OF THE INVENTION

In the present invention it was surprisingly found that a combination of specific anti-HER2 antibodies directed against an epitope between amino acids 342-652 of human HER2 or functional fragments or functional derivatives thereof with further HER2 inhibitors is particularly useful for therapeutic and diagnostic applications.

The epitope recognized by an antibody of the invention is preferably located in the domain III of human HER2. Preferably, the specific anti-HER2 antibodies are directed against an epitope between amino acids 342-510 of human HER2. Particularly preferred are humanized or human antibodies.

The specific antibody for use according to the present invention is characterized by six complementarity determining regions as described herein below:
a heavy chain complementarity determining region 1 (CDRH1) having the amino acid sequence as shown in SEQ ID NO: 1, or an amino acid sequence differing in 1 or 2 amino acids therefrom,
a heavy chain complementarity determining region 2 (CDRH2) having the amino acid sequence as shown in SEQ ID NO: 2, or an amino acid sequence differing in 1 or 2 amino acids therefrom,
a heavy chain complementarity determining region 3 (CDRH3) having the amino acid sequence as shown in SEQ ID NO: 3, or an amino acid sequence differing in 1 or 2 amino acids therefrom,
a light chain complementarity determining region 1 (CDRL1) having the amino acid sequence as shown in SEQ ID NO: 4, or an amino acid sequence differing in 1 or 2 amino acids therefrom,
a light chain complementarity determining region 2 (CDRL2) having the amino acid sequence as shown in SEQ ID NO: 5, or an amino acid sequence differing in 1 or 2 amino acids therefrom,
a light chain complementarity determining region 3 (CDRL3) having the amino acid sequence as shown SEQ ID NOs: 6 or 7, or an amino acid sequence differing in 1 or 2 amino acids therefrom.

Preferably, the antibody for use according to the invention comprises a heavy chain comprising the combination of CDRH1, CDRH2 and CDRH3 shown in table 1.

TABLE 1

| Seq | | Seq | | Seq | |
|---|---|---|---|---|---|
| ID | | ID | | ID | |
| No | CDR-H1 | No | CDR-H2 | No | CDR-H3 |
| 1 | NYGVS | 2 | IISGSGFTYYASWAKG | 3 | GVVPGYNAGGL |

According to the present invention, it is further preferred that the antibody comprises a light chain comprising the combination of CDRL1, CDRL2 and CDRL3 shown in table 2. It is understood that each line of this table represents one specific combination of a CDRL1, a CDRL2 and a CDRL3.

TABLE 2

| Seq | | Seq | | Seq | |
|---|---|---|---|---|---|
| ID | | ID | | ID | |
| No | CDR-L1 | No | CDR-L2 | No | CDR-L3 |
| 4 | QASQGISTALA | 5 | SASTLAS | 6 | QCTAAGSVSVGA |
| 4 | QASQGISTALA | 5 | SASTLAS | 7 | QSTAAGSVSVGA |

Most preferably, the antibody for use according to the invention comprises a heavy chain comprising the combination of CDRH1 as shown in SEQ ID No: 1, CDRH2 as shown in SEQ ID No: 2 and CDRH3 as shown in SEQ ID No: 3 and a light chain comprising the combination of CDRL1 as shown in SEQ ID No: 4, CDRL2 as shown in SEQ ID No: 5 and CDRL3 as shown in SEQ ID No: 6 or 7.

In the invention, it was found that antibodies having the complementarity determining regions as defined above show a uniquely strong apoptosis induction in partially HER2 resistant Breast Cancer Cell Line KPL-4 in contrast to trastuzumab. Induction of apoptosis is even stronger than of the positive control camptothecin.

Further in the KPL-4 in vivo breast cancer model, treatment with an antibody having the above defined CDRs in combination with trastuzumab led to significant tumor growth inhibition and was superior to the combination of trastuzumab and pertuzumab.

In addition, an antibody having the above defined CDRs specifically binds HER2 within a different epitope than the antibodies trastuzumab and pertuzumab and exhibits a different and unique mode of action. In in vitro assays an antibody having the above defined CDRs is capable of inducing apoptosis and strongly induces FcR mediated signaling pathways and consequently activation of antibody-dependent cellular toxicity (ADCC). Although trastuzumab and pertuzumab can also induce ADCC, there is no increase in ADCC efficiency when both agents were combined (Scheuer et al, 2009).

Therefore the present invention comprises an antibody having the above defined CDRs or a functional fragment or derivative thereof in combination with one or more second HER2 inhibitor(s), either in one single or two formulations, for the treatment of HER2 positive disease in a patient, who does not respond to a monotherapy with a HER2 inhibitor, or to successive monotherapies with at least two HER2 inhibitors, comprising co-administering to said patient an antibody having the above defined CDRs or a functional fragment or derivative thereof and at least one further HER2 inhibitor simultaneously or sequentially.

The invention further comprises an antibody having the above defined CDRs or a functional fragment or derivative thereof in combination with trastuzumab and/or pertuzumab, either in one single or two formulations, for the treatment of HER2 positive cancer or metastasis of HER2 positive cancer in a patient, who does not respond to a monotherapy with trastuzumab or pertuzumab, or to successive monotherapies with trastuzumab and pertuzumab, comprising co-administering to said patient an antibody having the above defined CDRs or a functional fragment or derivative thereof and trastuzumab or pertuzumab simultaneously or sequentially.

The invention further comprises an antibody having the above defined CDRs or a functional fragment or derivative thereof in combination with trastuzumab and/or pertuzumab for the treatment of HER2 positive cancer or metastasis of HER2 positive cancer in a patient, who does not respond to a first-line monotherapy with trastuzumab, comprising co-administering to said patient an antibody having the above defined CDRs or a functional fragment or derivative thereof and trastuzumab or pertuzumab simultaneously or sequentially

The invention further comprises an antibody having the above defined CDRs or a functional fragment or derivative thereof in combination with trastuzumab and/or pertuzumab for the treatment of HER2 positive cancer or metastasis of HER2 positive cancer in a patient, who does not respond to a first-line monotherapy with pertuzumab, comprising co-administering to said patient an antibody having the above defined CDRs or a functional fragment or derivative thereof and trastuzumab or pertuzumab simultaneously or sequentially.

The invention further comprises an antibody having the above defined CDRs or a functional fragment or derivative thereof in combination with trastuzumab and/or pertuzumab for the treatment of HER2 positive cancer or metastasis of HER2 positive cancer in a patient, who does respond neither to a monotherapy with trastuzumab nor to a monotherapy with pertuzumab, comprising co-administration of said patient an antibody having the above defined CDRs or a functional fragment or derivative thereof and trastuzumab or pertuzumab simultaneously or sequentially.

In the present invention it was surprisingly found that the combination of an antibody having the above defined CDRs or a functional fragment or derivative thereof with one or more agent(s) capable of blocking HER2 activity is superior to a combination of trastuzumab and pertuzumab.

Accordingly the present invention relates to an antibody having the above defined CDRs or a functional fragment or derivative thereof in combination with a second agent reducing the activity of HER2 for use in the prevention, alleviation or/and treatment of diseases, in particular diseases, associated with HER2 overexpression, amplification and/or hyperactivity.

The antibodies of the invention may be of various immunoglobulin (Ig) types, for example of the IgA-, IgD-, IgE-, IgG- or IgM-type, preferably of the IgG- or IgM-type including but not limited to the IgG1-, IgG2-, IgG3-, IgG4-, IgM1 and IgM2-type. In one preferred embodiment the antibody is of the IgG1 type.

As described above, the complementarity determining regions (CDRs) of an antibody may be flanked by framework regions. A heavy or light chain of an antibody containing three CDRs contains e.g. four framework regions.

Most preferably, the antibody of the invention comprises a heavy chain comprising four framework regions, wherein the combination of FR-H1, FR-H2, FR-H3 and FR-H4 is selected from those shown in table 3. It is understood that each line of this table represents one specific combination of FR-H1, FR-H2, FR-H3 and FR-H4.

TABLE 3

| | SEQ | | SEQ | | SEQ | | SEQ | |
|---|---|---|---|---|---|---|---|---|
| mAB | ID | | ID | | ID | | ID | |
| name | NO. | FR-H1 | NO. | FR-H2 | NO. | FR-H3 | NO. | FR-H4 |
| B100 | 8 | QSVEES | 17 | WVRQAP | 26 | RFTISKTS | 35 | WGQG |
| | | GGRLVT | | GKGLEY | | TTVDLKIT | | TLVTV |
| | | PGTPLT | | IG | | SPTTKDTA | | SS |
| | | LTCTVS | | | | TYFCAR | | |
| | | GFSLS | | | | | | |
| MAB | 9 | QVQLEE | 18 | WVRQAP | 27 | RFTISKDT | 36 | WGQG |
| 237 | | SGGRVV | | GKGLEY | | SKNTVVMQ | | TLVTV |
| | | QPGTSL | | VA | | MTSLRAED | | SS |
| | | RLSCAA | | | | TATYFCAR | | |
| | | SGFSLS | | | | | | |
| MAB | 10 | QVQLEE | 19 | WVRQAP | 28 | RFTISKDT | 37 | WGQG |
| 238 | | SGGRVV | | GKGLEY | | SKNTVVMQ | | TLVTV |
| | | QPGTSL | | VA | | MTSLRAED | | SS |
| | | RLSCAA | | | | TATYFCAR | | |
| | | SGFSLS | | | | | | |
| MAB | 11 | QVQLEE | 20 | WVRQAP | 29 | RFTISKDT | 38 | WGQG |
| 240 | | SGGRVV | | GKGLEY | | SKNTVVMQ | | TLVTV |
| | | QPGTSL | | VA | | MTSLRAED | | SS |
| | | RLSCAA | | | | TATYFCAR | | |
| | | SGFSLS | | | | | | |
| MAB | 12 | EEHLEE | 21 | WVRQAP | 30 | RFTISKDT | 39 | WGQG |
| 241 | | SGGRLV | | GRGLEY | | ARDSVYLQ | | TLVTV |
| | | KPGTSL | | VS | | MNSLRAED | | SS |
| | | RLSCTV | | | | TATYFCAR | | |
| | | SGFSLS | | | | | | |
| MAB | 13 | QVQLEE | 22 | WVRQAP | 31 | RFTISKDT | 40 | WGQG |
| 267 | | SGGRVV | | GKGLEY | | SKNTVVMQ | | TLVTV |
| | | QPGTSL | | VA | | MTSLRAED | | SS |
| | | RLSCAA | | | | TATYFCAR | | |
| | | SGFSLS | | | | | | |
| MAB | 14 | QVQLEE | 23 | WVRQAP | 32 | RFTISKDT | 41 | WGQG |
| 268 | | SGGRVV | | GKGLEY | | SKNTVVMQ | | TLVTV |
| | | QPGTSL | | VA | | MTSLRAED | | SS |
| | | RLSCAA | | | | TATYFCAR | | |
| | | SGFSLS | | | | | | |
| MAB | 15 | QVQLEE | 24 | WVRQAP | 33 | RFTISKDT | 42 | WGQG |
| 269 | | SGGRVV | | GKGLEY | | SKNTVVMQ | | TLVTV |
| | | QPGTSL | | VA | | MTSLRAED | | SS |
| | | RLSCAA | | | | TATYFCAR | | |
| | | SGFSLS | | | | | | |
| MAB | 16 | EEHLEE | 25 | WVRQAP | 34 | RFTISKDT | 43 | WGQG |
| 270 | | SGGRLV | | GRGLEY | | ARDSVYLQ | | TLVTV |
| | | KPGTSL | | VS | | MNSLRAED | | SS |
| | | RLSCTV | | | | TATYFCAR | | |
| | | SGFSLS | | | | | | |

Most preferably, the antibody of the invention comprises a light chain comprising four framework regions, wherein the combination of FR-L1, FR-L2, FR-L3 and FR-L4 is selected from those shown in table 4. It is understood that each line of this table represents one specific combination of FR-L1, FR-L2, FR-L3 and FR-L4.

TABLE 4

| | SEQ | | SEQ | | SEQ | | SEQ | |
|---|---|---|---|---|---|---|---|---|
| mAB | ID | | ID | | ID | | ID | |
| name | NO. | FR-L1 | NO. | FR-L2 | NO. | FR-L3 | NO. | FR-L4 |
| B100 | 44 | DIVMTQ | 53 | WYQQKP | 62 | GVSSRFKG | 71 | FGGG |
| | | TPASVS | | GQRPKL | | SGSGTQFT | | TEVVV |
| | | EPVGGT | | LIY | | LTISDLEC | | N |
| | | VTIKC | | | | ADAATYYC | | |
| MAB | 45 | DIQMTQ | 54 | WYQQKP | 63 | GVPSRFKG | 72 | FGGG |
| 237 | | SPSSLSA | | GQVPKL | | SGSGTEFT | | TEVVI |
| | | SVGDRI | | LIY | | LTISSLQA | | K |
| | | TITC | | | | EDVATYYC | | |
| MAB | 46 | DIVMTQ | 55 | WYQQKP | 64 | GVPSRFKG | 73 | FGQG |
| 238 | | SPSSVS | | GQAPKL | | SGSGTDFT | | TELVIK |
| | | ASVGDR | | LIY | | LTISSLQP | | |
| | | VTITC | | | | EDSATYYC | | |
| MAB | 47 | DIELTQ | 56 | WYQQKP | 65 | GVPSRFKG | 74 | FGGG |
| 240 | | SPSSVS | | GQAPKL | | SGSGTDFT | | TKVVI |
| | | ASVGDR | | LIY | | LTISSLQS | | E |
| | | VTITC | | | | EDSATYYC | | |
| MAB | 48 | DIQMTQ | 57 | WYQQKP | 66 | GVPSRFKG | 75 | FGGG |
| 241 | | SPSSLS | | GQVPKL | | SGSGTEFT | | TEVVI |
| | | ASVGDR | | LIY | | LTISSLQA | | K |
| | | ITITC | | | | EDVATYYC | | |
| MAB | 49 | DIQMTQ | 58 | WYQQKP | 67 | GVPSRFKG | 76 | FGGG |
| 267 | | SPSSLS | | GQVPKL | | SGSGTEFT | | TEVVI |
| | | ASVGDR | | LIY | | LTISSLQA | | K |
| | | ITITC | | | | EDVATYYC | | |
| MAB | 50 | DIVMTQ | 59 | WYQQKP | 68 | GVPSRFKG | 77 | FGQG |
| 268 | | SPSSVS | | GQAPKL | | SGSGTDFT | | TELVIK |
| | | ASVGDR | | LIY | | LTISSLQP | | |
| | | VTITC | | | | EDSATYYC | | |
| MAB | 51 | DIELTQ | 60 | WYQQKP | 69 | GVPSRFKG | 78 | FGGG |
| 269 | | SPSSVS | | GQAPKL | | SGSGTDFT | | TKVVI |
| | | ASVGDR | | LIY | | LTISSLQS | | E |
| | | VTITC | | | | EDSATYYC | | |
| MAB | 52 | DIQMTQ | 61 | WYQQKP | 70 | GVPSRFKG | 79 | FGGG |
| 270 | | SPSSLS | | GQVPKL | | SGSGTEFT | | TEVVI |
| | | ASVGDR | | LIY | | LTISSLQA | | K |
| | | ITITC | | | | EDVATYYC | | |

Furthermore, the antibody according to the invention may comprise
a) a heavy chain variable region (VH) that comprises the framework regions FR-H1, FR-H2, FR-H3, and FR-H4, wherein
   the FR-H1 region comprises an amino acid sequence selected from the group of SEQ ID NOs: 8-16,
   the FR-H2 region comprises an amino acid sequence selected from the group of SEQ ID NO: 17-25,
   the FR-H3 region comprises an amino acid sequence selected from the group of SEQ ID NOs: 26-34, and
   the FR-H4 region comprises an amino acid sequence selected from the group of SEQ ID NOs: 35-43; and
b) a light chain variable region (VL) that comprises the framework regions FR-L1, FR-L2, FR-L3, and FR-L4, wherein
   the FR-L1 region comprises an amino acid sequence selected from the group of SEQ ID NOs: 44-52,
   the FR-L2 region comprises an amino acid sequence selected from the group of SEQ ID NOs: 53-61,
   the FR-L3 region comprises an amino acid sequence selected from the group of SEQ ID NOs: 62-70, and
   the FR-L4 region comprises an amino acid sequence selected from the group of SEQ ID NOs: 71-79.

Additionally, the present invention also encompasses those antibodies that recognize the same epitope on human HER2 as a specific antibody characterized by the above heavy and/or light chain CDRs. Functional fragments and functional derivatives of those antibodies are also within the scope of the invention.

To determine the epitope on HER2 recognized by the antibody, chemically prepared arrays of protein sequence derived short peptides derived from the amino acid sequence of the extracellular domain of human HER2 can be used to locate and identify antibody epitopes (Reinicke W., Methods Mol. Biol. 2004, 248: 443-63). A further method to map the epitopes in the HER2 extracellular domain bound by the antibodies of the invention comprises Snaps/SELDI (Wang et al., Int. J. Cancer, 2001, June 15; 92 (6): 871-6) or a routine cross-blocking assay such as described in Antibodies, A Laboratory Manual, Cold Spring Harbor Laboratory, Ed Harlow and David Lane (1988) can be performed.

The antibody of the present invention is preferably a humanized or human antibody.

In a preferred embodiment of the invention, the human antibody comprises a heavy chain variable region (VH) as shown in any one of SEQ ID NOs. 80-88 or a sequence differing in 1 or 2 amino acids therefrom.

Furthermore, the human antibody of the invention preferably comprises a light chain variable region (VL) as shown in any one of SEQ ID NOs. 89-97 or a sequence differing in 1 or 2 amino acids therefrom.

Particularly preferred are human antibodies comprising a heavy chain variable region as shown in any one of SEQ ID NOs. 80-88 and a light chain variable region as shown in in any one of SEQ ID NOs. 89-97. In particular, it is preferred to use any one of antibodies disclosed below.

TABLE 5

| | SEQ | |
|---|---|---|
| mAB | ID | |
| name | NO. | Complete Heavy-chain VR sequence |
| B100 | 80 | QSVEESGGRLVTPGTPLTLTCTVSGFSLSNYGVSWVRQA |
| | | PGKGLEYIGIISGSGFTYYASWAKGRFTISKTSTTVDLK |
| | | ITSPTTKDTATYFCARGVVPGYNAGGLWGQGTLVTVSS |
| MAB | 81 | QVQLEESGGRVVQPGTSLRLSCAASGFSLSNYGVSWVRQ |
| 237 | | APGKGLEYVAIISGSGFTYYASWAKGRFTISKDTSKNTV |
| | | VMQMTSLRAEDTATYFCARGVVPGYNAGGLWGQGTLVTV |
| | | SS |
| MAB | 82 | QVQLEESGGRVVQPGTSLRLSCAASGFSLSNYGVSWVRQ |
| 238 | | APGKGLEYVAIISGSGFTYYASWAKGRFTISKDTSKNTV |
| | | VMQMTSLRAEDTATYFCARGVVPGYNAGGLWGQGTLVTV |
| | | SS |
| MAB | 83 | QVQLEESGGRVVQPGTSLRLSCAASGFSLSNYGVSWVRQ |
| 240 | | APGKGLEYVAIISGSGFTYYASWAKGRFTISKDTSKNTV |
| | | VMQMTSLRAEDTATYFCARGVVPGYNAGGLWGQGTLVTV |
| | | SS |
| MAB | 84 | EEHLEESGGRLVKPGTSLRLSCTVSGFSLSNYGVSWVRQ |
| 241 | | APGRGLEYVSIISGSGFTYYASWAKGRFTISKDTARDSV |
| | | YLQMNSLRAEDTATYFCARGVVPGYNAGGLWGQGTLVTV |
| | | SS |
| MAB | 85 | QVQLEESGGRVVQPGTSLRLSCAASGFSLSNYGVSWVRQ |
| 267 | | APGKGLEYVAIISGSGFTYYASWAKGRFTISKDTSKNTV |
| | | VMQMTSLRAEDTATYFCARGVVPGYNAGGLWGQGTLVTV |
| | | SS |
| MAB | 86 | QVQLEESGGRVVQPGTSLRLSCAASGFSLSNYGVSWVRQ |
| 268 | | APGKGLEYVAIISGSGFTYYASWAKGRFTISKDTSKNTV |
| | | VMQMTSLRAEDTATYFCARGVVPGYNAGGLWGQGTLVTV |
| | | SS |
| MAB | 87 | QVQLEESGGRVVQPGTSLRLSCAASGFSLSNYGVSWVRQ |
| 269 | | APGKGLEYVAIISGSGFTYYASWAKGRFTISKDTSKNTV |
| | | VMQMTSLRAEDTATYFCARGVVPGYNAGGLWGQGTLVTV |
| | | SS |
| MAB | 88 | EEHLEESGGRLVKPGTSLRLSCTVSGFSLSNYGVSWVRQ |
| 270 | | APGRGLEYVSIISGSGFTYYASWAKGRFTISKDTARDSV |
| | | YLQMNSLRAEDTATYFCARGVVPGYNAGGLWGQGTLVTV |
| | | SS |

TABLE 6

| | SEQ | |
|---|---|---|
| mAB | ID | |
| name | NO. | Complete κ-Light chain VR sequence |
| B100 | 89 | DIVMTQTPASVSEPVGGTVTIKCQASQGISTALAWYQQK |
| | | PGQRPKLLIYSASTLASGVSSRFKGSGSGTQFTLTISDL |
| | | ECADAATYYCQCTAAGSVSVGAFGGGTEVVVN |
| MAB | 90 | DIQMTQSPSSLSASVGDRITITCQASQGISTALAWYQQK |
| 237 | | PGQVPKLLIYSASTLASGVPSRFKGSGSGTEFTLTISSL |
| | | QAEDVATYYCQCTAAGSVSVGAFGGGTEVVIK |
| MAB | 91 | DIVMTQSPSSVSASVGDRVTITCQASQGISTALAWYQQK |
| 238 | | PGQAPKLLIYSASTLASGVPSRFKGSGSGTDFTLTISSL |
| | | QPEDSATYYCQCTAAGSVSVGAFGQGTELVIK |
| MAB | 92 | DIELTQSPSSVSASVGDRVTITCQASQGISTALAWYQQK |
| 240 | | PGQAPKLLIYSASTLASGVPSRFKGSGSGTDFTLTISSL |
| | | QSEDSATYYCQCTAAGSVSVGAFGGGTKVVIE |
| MAB | 93 | DIQMTQSPSSLSASVGDRITITCQASQGISTALAWYQQK |
| 241 | | PGQVPKLLIYSASTLASGVPSRFKGSGSGTEFTLTISSL |
| | | QAEDVATYYCQCTAAGSVSVGAFGGGTEVVIK |
| MAB | 94 | DIQMTQSPSSLSASVGDRITITCQASQGISTALAWYQQK |
| 267 | | PGQVPKLLIYSASTLASGVPSRFKGSGSGTEFTLTISSL |
| | | QAEDVATYYCQSTAAGSVSVGAFGGGTEVVIK |
| MAB | 95 | DIVMTQSPSSVSASVGDRVTITCQASQGISTALAWYQQK |
| 268 | | PGQAPKLLIYSASTLASGVPSRFKGSGSGTDFTLTISSL |
| | | QPEDSATYYCQSTAAGSVSVGAFGQGTELVIK |
| MAB | 96 | DIELTQSPSSVSASVGDRVTITCQASQGISTALAWYQQK |
| 269 | | PGQAPKLLIYSASTLASGVPSRFKGSGSGTDFTLTISSL |
| | | QSEDSATYYCQSTAAGSVSVGAFGGGTKVVIE |
| MAB | 97 | DIQMTQSPSSLSASVGDRITITCQASQGISTALAWYQQK |
| 270 | | PGQVPKLLIYSASTLASGVPSRFKGSGSGTEFTLTISSL |
| | | QAEDVATYYCQSTAAGSVSVGAFGGGTEVVIK |

Particularly preferred is a human antibody (MAB270) comprising a heavy chain comprising a CDRH1 as shown in SEQ ID NO: 1, a CDRH2 as shown in SEQ ID NO: 2 and a CDRH3 as shown in SEQ ID NO: 3 and a light chain comprising a CDRL1 as shown in SEQ ID NO: 4, a CDRL2 as shown in SEQ ID NO: 5 and a CDRL3 as shown in SEQ ID NO: 7. Also suitable are human antibodies, wherein one or more of the CDRs differ in 1 or 2 amino acids or antibodies recognizing the same epitope on human HER2.

In a particularly preferred embodiment, the human antibody comprises a heavy chain variable region according to SEQ ID NO: 88 and a light chain variable region according to SEQ ID NO: 97. Also suitable are human antibodies wherein the sequences of the variable region of the heavy chain and/or the light chain differ in 1 or 2 amino acids from those shown in SEQ ID NOs. 88 and 97.

A monoclonal antibody according to the invention can be rabbit antibody. In a preferred embodiment, the antibody of the invention is a rabbit/human chimeric antibody. In a further preferred version, the antibody is a humanized antibody.

The present invention also encompasses an antibody that specifically binds to HER2, or a fragment or derivative thereof or a polypeptide that contains at least a portion of said antibody that is sufficient to confer HER2 binding specificity, wherein said antibody binds to the human Fc receptor and induces FcR mediated signaling pathways.

Preferably, the antibodies according to the invention show an increased induction of FcR mediated signaling pathway, when compared to trastuzumab or pertuzumab.

In KPL-4 cells, the antibodies according to the invention show a stimulation of apoptosis that is preferably 100% higher than untreated cells, more preferably 110% higher than untreated cells and most preferably 120% higher than untreated cells. This reflects a much higher potency than the HER2 antibody trastuzumab. Trastuzumab exhibits a comparable increase of apoptosis that is 69% of untreated cells (FIG. 1).

This increased activity of antibodies according to the invention in comparison to trastzumab and pertuzumab used in cancer therapy clearly shows its superiority and outstanding potential for the use in the treatment of HER2-mediated diseases.

The term “rabbit” according to the invention means an animal of the members of the taxonomic order Lagomorpha, which includes the families (hares and rabbits) and Ochotonidae (pikas), preferably of genus Oryctolagus. The term “antibody” encompasses the various forms of antibody structures including, but not being limited to, whole antibodies and antibody fragments as long as it shows the properties according to the invention.

The term “rabbit monoclonal antibody” according to the invention means a monoclonal antibody produced by immunizing a rabbit and isolated from an antigen producing cell of said rabbit as well as such an antibody which is further modified, preferably a humanized antibody, a chimeric antibody, a fragment thereof, or a further genetically engineered and recombinant produced antibody as long as the characteristic properties according to the invention are retained. Preferably the antibody is from a B cell or a rabbit hybridoma cell of said rabbit.

The term “antibody producing cell” according to the invention means a rabbit B cell which produce antibodies, preferably a B cell or rabbit hybridoma cell.

“Native antibodies” are usually heterotetrameric glycoproteins composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The “variable region (or domain)” (variable region of a light chain (VL), variable region of a heavy chain (VH) as used herein denotes each of the pair of light and heavy chain regions which are involved directly in binding the antibody to the antigen. The variable light and heavy chain regions have the same general structure and each region comprises four framework (FR) regions whose sequences are widely conserved, connected by three complementary determining regions, CDRs.

The term “antigen-binding portion of an antibody” refers to the amino acid residues of an antibody which are responsible for antigen-binding. The antigen-binding portion of an antibody comprises preferably amino acid residues from the “complementary determining regions” or “CDRs”. The CDR sequences are defined according to Kabat et al, Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable region.

For example, a heavy chain variable region may include a single amino acid insert (residue 52a according to Kabat) after residue 52 of H2 and inserted residues (e.g. residues 82a, 30 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The “constant domains (constant parts)” are not involved directly in binding of an antibody to an antigen, but exhibit e.g. also effector functions. The heavy chain constant region that corresponds to human IgGI is called γl chain. The heavy chain constant region that corresponds to human IgG3 is called γ3 chain. Human constant γ heavy chains are described in detail by Kabat, E. A. et al., Sequences of Proteins of Immunological Interest, 5th ed., Public Health Service, National Institutes of Health, Bethesda, Md. (1991), and by Brueggemann, M., et al., J. Exp. Med. 166 (1987) 1351-1361; Love, T. W., et al., Methods Enzymol. 178 (1989) 515-527. Constant domains of IgG1 or IgG3 type are glycosylated at Asn297. “Asn 297” according to the invention means amino acid asparagine located at about position 297 in the Fc region; based on minor sequence variations of antibodies, Asn297 can also be located some amino acids (usually not more than +3 amino acids) upstream or downstream.

The term “antibody effector function(s)” or “effector function” as used herein refers to a function mediated by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Typical effector functions are ADCC, ADCP and CDC. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2; diabodies; linear antibodies; single-chain antibody molecules (e.g. scFv); and multispecific antibodies formed from antibody fragments.

“Antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FCYRIII. FCR expression on hematopoietic cells is summarized in Table 3 on page 464 of Ravetch, and Kinet, Annu. Rev. Immunol 9 (1991) 457-492. The term “Antibody-dependent cellular phagocytosis” and “ADCP” refer to a process by which antibody-coated cells are internalized, either in whole or in part, by phagocytic immune cells (e.g., macrophages, neutrophils and dendritic cells) that bind to an immunoglobulin Fc region.

Clq” is a polypeptide that includes a binding site for the Fc region of an immunoglobulin. Clq together with two serine proteases, Clr and Cls, forms the complex CI, the first component of the complement dependent cytotoxicity (CDC) pathway. Fluman Clq can be purchased commercially from, e.g. Quidel, San Diego, Calif.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA5, and IgA2. The heavy chain constant domains that correspond to the different classes of immunoglobulins are called a, 6, s, y, and p, respectively.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions.

Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, Md. (1991).

A “variant Fc region” comprises an amino acid sequence which differs from that of a “native” or “wildtype” sequence Fc region by virtue of at least one “amino acid modification” as herein defined.

The term “Fc-variant” as used herein refers to a polypeptide comprising a modification in the Fc domain. For all positions discussed in the present invention, numbering is according to the EU index. The EU index or EU index as in Kabat or EU numbering scheme refers to the numbering of the EU antibody (Edelman, et al., Proc Natl Acad Sei USA 63 (1969) 78-85, hereby entirely incorporated by reference.) The modification can be an addition, deletion, or substitution. Substitutions can include naturally occurring amino acids and non-naturally occurring amino acids. Variants may comprise non-natural amino acids.

The term “Fc region-containing polypeptide” refers to a polypeptide, such as an antibody or immunoadhesin (see definitions below), which comprises an Fc region.

The terms “Fc receptor” or “FcR” are used to describe a receptor that binds to the Fc region of an antibody. A FcR which binds an IgG antibody (a gamma receptor) includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain, (see review in Daeron, M., Annu. Rev. Immunol. 15 (1997) 203-234). FcRs are reviewed in Ravetch, and Kinet, Annu. Rev. Immunol 9 (1991) 457-492; Capel, et al., Immunomethods 4 (1994) 25-34; and de Flaas, et al., J. Lab. Clin. Med. 126 (1995) 330-41. Other FcRs, including those to be identified in the future are encompassed by the term “FcR” herein. The term also includes the neonatal receptor, FcRn, which is responsible for the transfer of maternal IgGs to the fetus (Guyer, et al., J. Immunol. 117 (1976) 587 and Kim, et al., J. Immunol. 24 (1994) 249). By “IgG Fc ligand” as used herein is meant a molecule, preferably a polypeptide, from any organism that binds to the Fc region of an IgG antibody to form an Fc/Fc ligand complex. Fc ligands include but are not limited to FcyRs, FcyRs, FcyRs, FcRn, Clq, C3, mannan binding lectin, mannose receptor, staphylococcal protein A, streptococcal protein G, and viral FcyR. Fc ligands also include Fc receptor homologs (FcRH), which are a family of Fc receptors that are homologous to the FcyRs (Davis, et al., Immunological Reviews 190 (2002) 123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex By “Fc gamma receptor”, “FcyR” or “FcgammaR” as used herein is meant any member of the family of proteins that bind the IgG antibody Fc region and is encoded by an FcyR gene. In humans this family includes but is not limited to FcyRI (CD64), including isoforms FcyRIA, FcyRIB, and FcyRIC; FcyRII (CD32), including isoforms FcyRIIA (including allotypes H131 and R131), FcyRIIB (including FcyRIIB-1 and FcyRIIB-2), and FcyRIIc; and FcyRII) (CD 16), including isoforms FcyRIIIA (including allotypes VI 58 and F158) and FcyRI11b (including allotypes FcyRIIB-NAI and FcyRIIB-NA2) (Jefferis, et al., Immunol Lett 82(2002) 57-65, entirely incorporated by reference), as well as any undiscovered human FcyRs or FcyR isoforms or allotypes. An FcyR may be from any organism, including but not limited to humans, mice, rats, rabbits, and monkeys. Mouse FcyRs include but are not limited to FcyRI (CD64), FcyRII (CD32), FcyRII) (CD 16), and FCYRIII-2 (CD 16-2), as well as any undiscovered mouse FcyRs or FcyR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin.

An “antibody that binds to the same epitope” as a reference antibody refers to an antibody that blocks binding of the reference antibody to its target antigen in a competition assay. Possible epitope overlapping of two antibodies binding to the same target antigen can be detected with the help of a competitive test system. For this purpose, for example with the help of an enzyme immunoassay, there is tested the extent to which the new antibody competes with the known antibody for the binding to an immobilized target antigen. For this purpose, an appropriately immobilized target antigen is incubated with the known antibody in labeled form and an excess of the antibody in question. By detection of the bound labeling there can easily be ascertained the extent to which the antibody in question can displace the known antibody from the binding site (=epitope). If there is a displacement of more than 10%, preferably of more than 20%, at the same concentration or at higher concentrations, preferably in the case of 10-fold excess of the antibody in question, referred to the known antibody, then an epitope overlapping is present. That means that the antibody in question binds to the same epitope as the known antibody.

Immunoassays are well known to the skilled artisan. Methods for carrying out such assays as well as practical applications and procedures are summarized in related textbooks. Examples of related textbooks are Tijssen, R, Preparation of enzyme-antibody or other enzyme-macromolecule conjugates, in: Practice and theory of enzyme immunoassays, Burdon, R. H. and v. Knippenberg, P. H. (eds.), Elsevier, Amsterdam (1990) pp. 221-278; and various volumes of Methods in Enzymology, Colowick, S. P. and Caplan, N. O.(eds.), Academic Press, dealing with immunological detection methods, especially volumes 70, 73, 74, 84, 92 and 121.

The term “epitope” as used within this application denotes a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. Depending on the size of the antigen to which the epitope belongs, more than one epitope per antigen may be available resulting likewise in the possibility of more than one antibody binding site (=epitope) per antigen.

An “immunoconjugate” means an antibody conjugated to one or more cytotoxic agents, such as a chemotherapeutic agent, a drug, a growth inhibitory agent, a toxin, another antibody or a radioactive isotope.

“Antibody fragments” comprise a portion of a full-length antibody, preferably the variable regions thereof, or at least the antigen binding site thereof. Examples of antibody fragments include diabodies, Fab fragments, and single-chain antibody molecules. scFv antibodies are, e.g., described in Fluston, J. S., Methods in Enzymol. 203 (1991) 46-88.

The terms “monoclonal antibody” or “monoclonal antibody composition” as used herein refer to a preparation of antibody molecules of a single amino acid composition.

The term “humanized antibody” or “humanized version of an antibody” refers to antibodies for which both heavy and light chains are humanized as a result of antibody engineering. A humanized chain is typically a chain in which the V-region amino acid sequence has been changed so that, analyzed as a whole, is closer in homology to a human germline sequence than to the germline sequence of the species of origin. Humanization assessment is based on the resulting amino acid sequence and not on the methodology per se.

The terms “specifically binding, against target, or anti-target antibody”, as used herein, refer to binding of the antibody to the respective antigen (target) or antigen-expressing cell, measured by ELISA, wherein said ELISA preferably comprises coating the respective antigen to a solid support, adding said antibody under conditions to allow the formation of an immune complex with the respective antigen or protein, detecting said immune complex by measuring the Optical Density values (OD) using a secondary antibody binding to an antibody according to the invention and using a peroxidase-mediated color development.

The term “antigen” according to the invention refers to the antigen used for immunization or a protein comprising said antigen as part of its protein sequence. For example, for immunization a fragment of the extracellular domain of a protein (e.g. the first 20 amino acids) can be used and for detection/assay and the like the extracellular domain of the protein or the full length protein can be used.

The term “specifically binding” or “specifically recognized” herein means that an antibody exhibits appreciable affinity for an antigen and, preferably, does not exhibit significant cross-reactivity.

“Appreciable” binding affinity includes binding with an affinity of at least 10⁻⁷ M, specifically at least 10⁻⁸M, more specifically at least 10⁻⁹M, or even yet more specifically at least 10⁻¹⁰ M.

An antibody that “does not exhibit significant cross-reactivity” is one that will not appreciably bind to an undesirable other protein. Specific binding can be determined according to any art-recognized means for determining such binding, e.g. by competitive binding assays such as ELISA. All protein terms as used herein refers to the human proteins. If a protein from another species is meant, this is explicitly mentioned.

The term “cancer” as used herein may be, for example, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers. Preferably such cancer is a breast cancer, colon cancer, lung cancer, or pancreatic cancer

In the context of this invention, additional other cytotoxic, chemotherapeutic or anti-cancer agents, or compounds that enhance the effects of such agents may be used in the combination treatment of HER2 positive cancer or metastasis of HER2 positive cancer with an antibody having the above defined CDRs.

Such agents include, for example: alkylating agents or agents with an alkylating action, such as cyclophosphamide (CTX; e.g. Cytoxan®)

In the context of this invention, an anti-hormonal agent may be used in the combination treatment of HER2 positive cancer or metastasis of HER2 positive cancer with an antibody having the above defined CDRs. As used herein, the term “anti-hormonal agent” includes natural or synthetic organic or peptidic compounds that act to regulate or inhibit hormone action on tumors.

In the context of this invention, additional anti-proliferative agents may be used in the combination treatment of HER2 positive cancer or metastasis of HER2 positive cancer with an antibody having the above defined CDRs.

In the context of this invention, an effective amount of ionizing radiation may be carried out and/or a radiopharmaceutical may be used in the combination treatment of HER2 positive cancer or metastasis of HER2 positive cancer with an antibody having the above defined CDRs.

The present invention further relates to a pharmaceutical composition comprising a pharmaceutically acceptable carrier and a therapeutically effective amount of an antibody having the above defined CDRs in combination with a second agent according to the invention. Said pharmaceutical composition can be administered to a patient in a method of treating an HER-2 mediated disease according to the invention.

The present invention also encompasses the administration of the pharmaceutical composition to a patient. As is well-known in the medical arts, dosages for any one patient depend upon many factors including the patient's size, body surface and area, age, the particular compound to be administered, sex, time and route of administration, general health and other drugs being administered concurrently. Depending on the type and severity of the condition to be treated, about 1 μg/kg to 15 mg/kg of the active ingredient may be administered to a patient in need thereof, e.g. by one or more separate administrations or by continuous infusion. A typical daily dosage might range from about 1 μg/kg to about 100 mg/kg, depending on the factors mentioned above. For repeated administrations over several days or longer, depending on the condition to be treated, the treatment is sustained until a desired suppression of the disease or the symptoms occurs. The composition may be administered by any suitable route, for example by parental, subcutaneous, intranasal, intravascular, intravenous, intraarterial or intrathecal injection or infusion. Progress can be monitored by periodic assessment. The compositions of the inventions may be administered locally or systemically. Preparations for parental administration include sterile aqueous or non-aqueous solutions, suspensions and emulsions. Examples of non-aqueous solvents are propylenes, glycol, polyethylene glycol, vegetable oils such as olive oil, and injectable organic esters such as ethyl-oleate. Aqueous carriers include water, alcoholic/aqueous solutions, emulsions or suspensions, including saline and buffered media. Parental vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present, such as for example antimicrobials, antioxidants, chelating agents and inert gases, and the like.

The present invention relates also to a kit which comprises an antibody having the above defined CDRs and a second agent and a package insert instructing the user to co-administer an antibody having the above defined CDRs and a second agent to a patient suffering from HER2 positive cancer who does not respond to a monotherapy with the second agent.

The following examples and figures including the experiments conducted and the results achieved are provided for illustration only and are not to be construed as limiting the teachings of the present invention.

### FIGURES

FIG. 1. Apoptosis induction was measured as Annexin V staining after incubation of KPL-4 cells with the antibody Trastuzumab or MAB270. Shown are the Annexin V-positive, apoptotic cells compared to the positive control camptothecin (positive control set as 100%). Data are presented as mean±SD (n=3).

FIG. 2. Tumor volume (cm3) of individual mice 33 days (A) and 62 days (B) after tumor cell injection (treatment started on day 15). KPL-4 xenograft mice were treated with vehicle (circles), co-administration of Trastuzumab and Pertuzumab (squares), monotherapy of MAB270 (triangle) or co-administration of MAB270 and Trastuzumab (reversed triangle). Mean±SD is indicated. *, P<0.05 (Mann Whitney test).

FIG. 3. Individual data points of tumor volume (cm3) before treatment (day 15) and at the end of the experiment (day 78) are shown for the co-administration of Trastuzumab/Pertuzumab and the co-administration of/Trastuzumab. Mice showing tumor regression are highlighted.

FIG. 4. Kaplan-Meier survival curves of tumor bearing mice treated as indicated. Mice were sacrificed when they reached a tumor volume of 1.5 cm3, got moribund or lost body weight. ***, P<0,0001 for MAB270/Trastuzumab vs control and *, P=0.0224 for MAB270 vs control (Log-rank test)

FIG. 5. Epitope Mapping of Pertuzumab, Trastuzumab and B100. B100 anti-HER2 was analyzed in a binding ELISA on different domains of the ECD of HER2. B100 binds to a domain different from trastuzumab and pertuzumab.

### EXAMPLE 1: APOPTOSIS INDUCTION ON KPL-4 CELL LINE

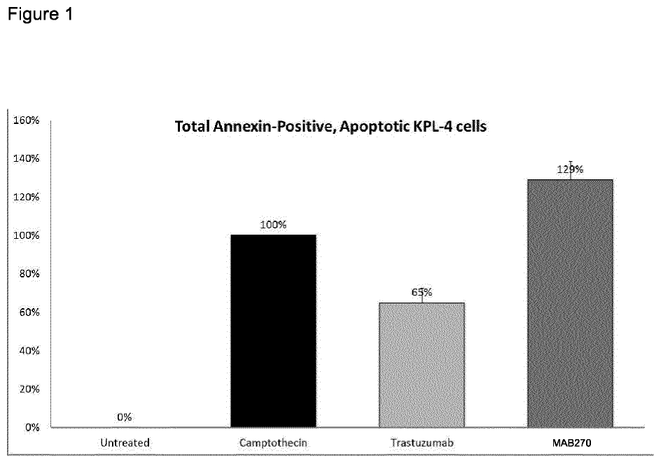

To analyze the induction of apoptosis, an in vitro Annexin V staining assay after was performed. KPL-4 cells were seeded in 2 ml medium (4×104 cells/well) and incubated for 72 h before treatment, followed by incubation with 5 μg/ml antibody or assay-medium as control for 72 h at 37° C. As positive control 2 mM camptothecin was used (incubation for 24 h). Cells and supernatant were harvested, centrifuged and washed with PBS. Afterwards cells were re-suspended in precooled binding buffer (0,1M HEPES, 1.4 M NaCl, 25 mM CaCl2)) and mixed with Annexin (Annexin V FITC-conjugate from Immunotools) and incubated for 20 min on ice/dark. For staining of dead cells DRAQ7 was used (7 min incubation on ice/dark). Annexin staining was determined by flow cytometry analysis. The tested MAB 270 shows a uniquely strong apoptosis induction in contrast to trastuzumab. The antibody is capable of inducing apoptosis in a higher number of KPL-4 cells compared to the positive control camptothecin, whereas trastuzumab shows less activity (FIG. 1).

### EXAMPLE 2: ANTITUMOR ACTIVITY IN A KPL-4 BREAST CANCER XENOGRAFT MODEL

To evaluate the antitumor activity of MAB270 alone and in combination with Trastuzumab compared to Trastuzumab in combination with Pertuzumab, a breast cancer xenograft model was applied. Female SCID beige mice (Charles River Laboratories) aged 6-8 weeks were intra mammary fat pad injected with 3×10⁶ KPL-4 tumor cells. Tumor cell injection corresponds to day 0 of the experiment. Tumor-bearing mice were stratified according to tumor size of approximately 100 cm³ (n=8 for each group) and treatment started at day 15. All tested antibodies were provided in the same buffer (20 mM Histidin, 140 mM NaCl, pH 6.0). Vehicle group received reference buffer intraperitoneally (i.p.) once weekly. MAB270 was administered i.p. at a loading dose of 30 mg/kg, followed by a maintenance dose of 15 mg/kg once weekly. The combination of MAB270 and Trastuzumab or the combination of Trastuzumab and Pertuzumab were given in the same dose regimen and schedule as the monotherapy. Throughout the experiment the body weight of the animals and the tumor volume were measured twice per weak. Mice were sacrificed when they reached a tumor volume of 1.5 cm³, got moribund or lost body weight

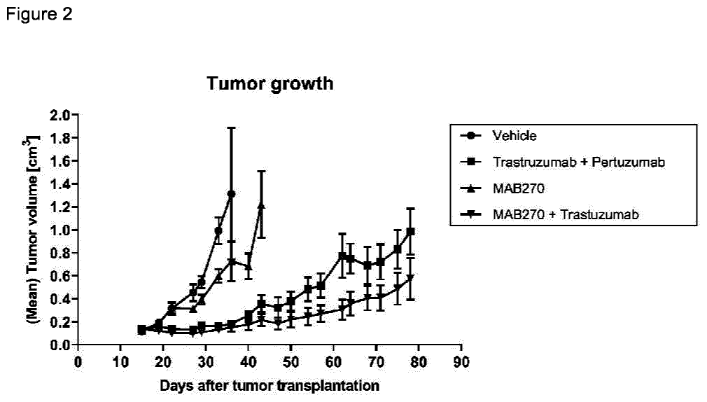

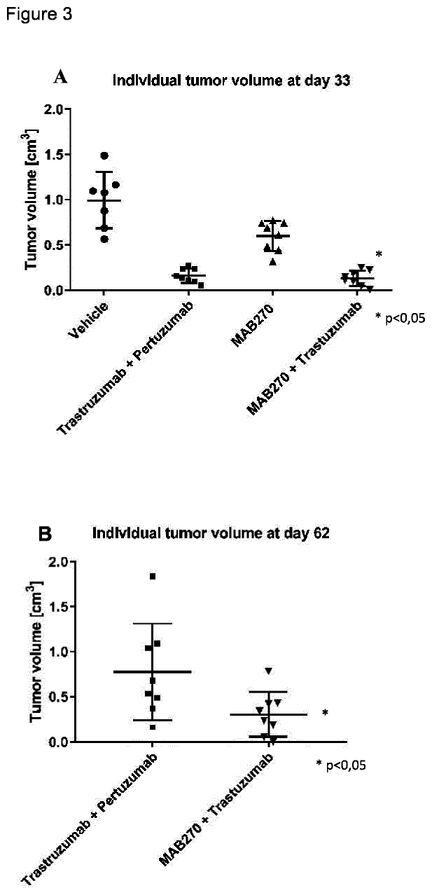

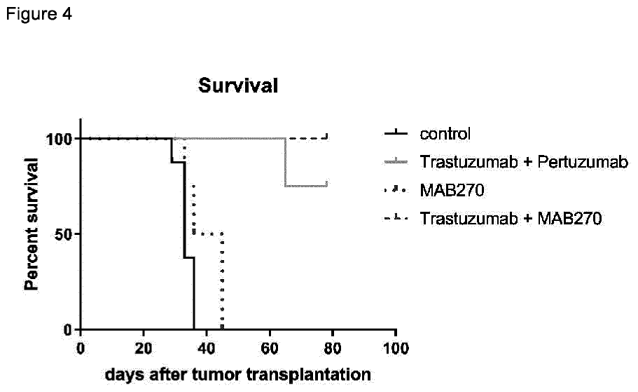

On day 33, tumors in control animals reached a mean tumor volume of 1.0 cm³ and individual mice had to be sacrificed from day 33 on. Treatment with only MAB270 at 15 mg/kg once per week for 3 cycles (initially 30 mg/kg for the first cycle) significantly reduced tumor growth compared to the vehicle group (FIG. 2) and resulted in a significant longer survival until mice had to be sacrificed due to tumor volume (FIG. 4). Treatment with combination therapy significantly inhibited tumor growth compared to control (FIG. 2). A combination of MAB270 and trastuzumab (each 15 mg/kg once a week for 8 cycles; initially 30 mg/kg) was superior compared to control and MAB270 monotherapy (FIG. 2) and resulted in a longer survival until mice had to be sacrificed due to tumor volume. In addition, this combination therapy showed significantly improved efficacy compared to the combination trastuzumab and pertuzumab (FIG. 3). In the group with the treatment MAB270 plus trastuzumab, individual mice showed tumor regression, whereas in the group treated with trastuzumab plus pertuzumab individual mice had to be sacrificed due to their tumor volume (FIG. 4).

### EXAMPLE 3: EPITOPE MAPPING

Binding of anti-HER2 monoclonal antibodies trastuzumab, pertuzumab and B100 to full length extracellular domain of HER2 or to different sub-domains of HER2 was tested in a biochemical ELISA. Recombinant Her2 domains (full length ECD purchased from Biozol, ECD domains produced at Mab Discovery), were coated on a 384-well Nunc™ MaxiSorp™ plate at optimized concentrations (0.25-1 mg/ml) in PBS for one hour at room temperature. After washing three times with wash buffer (PBS, 0.1% Tween), plates were blocked with PBS, 2% BSA, 0.05% Tween for one hour at room temperature. Plates were washed again three time with wash buffer and antibodies at a concentration of 10 μg/ml in PBS, 0.5% BSA, 0.05% Tween were incubated for one hour at room temperature. After 3 washes in wash buffer, wells were incubated with 12.5 μl of a 1:5000 dilution of anti-human peroxidase-linked, species specific F(ab)2 Fragment from goat (AbD Serotec) in ELISA buffer for one hour at room temperature. Wells were washed six times with wash buffer and 15 μl/well TMB substrate solution (Invitrogen) were added. After 10 minutes at room temperature 15 μl Stop solution (1M HCl) were added per well and absorbance at 450 and 620 nm wavelength was measured using a Tecan M1000 microplate reader.

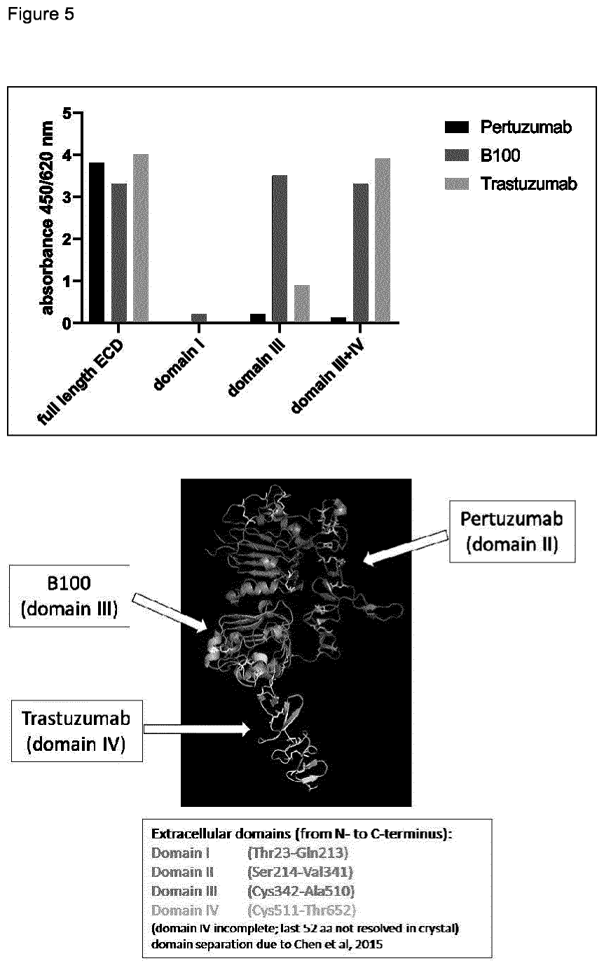

All antibodies show binding to the HER2 full-length extracellular domain (ECD). For trastuzumab, which is described to be a sub-domain IV binder, binding could only be observed to the fusion-protein containing sub-domain III and IV. Trastuzumab binds neither to sub-domain I, nor to sub-domain III. Pertuzumab does not bind to one of the tested HER2 domains I, Ill or IV. Pertuzumab is described to be a domain II binder. Due to the results of the shown in FIG. 5, B100 antibody can be considered as a sub-domain III binder. Binding could be observed for the full-length ECD, domain III and the domain III-IV fusion protein, but not to the sub-domain I. The ELISA binding assay clearly shows that B100 is binding to a new epitope of HER2, different to the sub-domains of pertuzumab and trastuzumab.

1. A pharmaceutical composition or kit comprising, either in a single formulation or in two separate formulations,
a monoclonal anti-HER2 antibody or a functional fragment or derivative thereof and a second HER2 inhibitor, and optionally one or more pharmaceutically acceptable carriers, additives or further active agents,
wherein the anti-HER2 antibody is an antibody comprising:
a heavy chain complementarity determining region 1 (CDRH1) having the amino acid sequence as shown in SEQ ID NO: 1, or an amino acid sequence differing in 1 or 2 amino acids therefrom,
a heavy chain complementarity determining region 2 (CDRH2) having the amino acid sequence as shown in SEQ ID NO: 2, or an amino acid sequence differing in 1 or 2 amino acids therefrom,
a heavy chain complementarity determining region 3 (CDRH3) having the amino acid sequence as shown in SEQ ID NO: 3, or an amino acid sequence differing in 1 or 2 amino acids therefrom,
a light chain complementarity determining region 1 (CDRL1) having the amino acid sequence as shown in SEQ ID NO: 4, or an amino acid sequence differing in 1 or 2 amino acids therefrom,
a light chain complementarity determining region 2 (CDRL2) having the amino acid sequence as shown in SEQ ID NO: 5, or an amino acid sequence differing in 1 or 2 amino acids therefrom, and
a light chain complementarity determining region 3 (CDRL3) having the amino acid sequence as shown SEQ ID NOs: 6 or 7, or an amino acid sequence differing in 1 or 2 amino acids therefrom,
or an antibody recognizing an epitope within amino acids 342-652 of human HER2.

2. The pharmaceutical composition or kit of claim 1, wherein the anti-HER2 antibody is an antibody comprising a CDRH1 as shown in SEQ ID NO: 1, a CDRH2 as shown in SEQ ID NO: 2, a CDRH3 as shown in SEQ ID NO: 3, a CDRL1 as shown in SEQ ID NO: 4, a CDRL2 as shown in SEQ ID NO: 5 and a CDRL3 as shown in SEQ ID NO: 6 or 7.

3. The pharmaceutical composition or kit of claim 1, wherein the anti-HER2 antibody comprises
a) a heavy chain variable region (VH) that comprises the framework regions FR-H1, FR-H2, FR-H3, and FR-H4, wherein
the FR-H1 region comprises an amino acid sequence selected from the group of SEQ ID NOs: 8-16,
the FR-H2 region comprises an amino acid sequence selected from the group of SEQ ID NOs: 17-25,
the FR-H3 region comprises an amino acid sequence selected from the group of SEQ ID NOs: 26-34, and
the FR-H4 region comprises an amino acid sequence selected from the group of SEQ ID NO: 35-43, and
b) a light chain variable region (VL) that comprises the framework regions FR-L1, FR-L2, FR-L3, and FR-L4, wherein
the FR-L1 region comprises an amino acid sequence selected from the group of SEQ ID NOs: 44-52,
the FR-L2 region comprises an amino acid sequence selected from the group of SEQ ID NOs: 53-61,
the FR-L3 region comprises an amino acid sequence selected from the group of SEQ ID NOs: 62-70, and
the FR-L4 region comprises an amino acid sequence selected from the group of SEQ ID NOs: 71-79.

4. The pharmaceutical composition or kit of claim 1, wherein the anti-HER2 antibody comprises a heavy chain variable region (VH) as shown in any one of SEQ ID NOs. 80-88 or a sequence differing in 1 or 2 amino acids therefrom, and/or a light chain variable region (VL) as shown in any one of SEQ ID NOs. 89-97 or a sequence differing in 1 or 2 amino acids therefrom.

5. The pharmaceutical composition or kit of claim 1, wherein the anti-HER2 antibody is a humanized or human antibody.

6. The pharmaceutical composition or kit of claim 1, wherein the second HER2 inhibitor is an anti-HER2 antibody binding within a different epitope on human HER2.

7. The pharmaceutical composition or kit of claim 1, wherein the second HER2 inhibitor is trastuzumab or pertuzumab.

8. The pharmaceutical composition or kit of claim 1, in combination with one or more further cytotoxic, chemotherapeutic or anti-cancer agents.

9. (canceled)

10. (canceled)

11. (canceled)

12. (canceled)

13. A method for prevention or treatment of a disease associated with HER2 overexpression, amplification and/or hyperactivity, comprising co-administering to a patient in need thereof simultaneously or sequentially a therapeutically effective amount of a monoclonal anti-HER2 antibody or a functional fragment or derivative thereof and at least one second HER2 inhibitor
wherein the anti-HER2 antibody is an antibody comprising:
a heavy chain complementarity determining region 1 (CDRH1) having the amino acid sequence as shown in SEQ ID NO: 1, or an amino acid sequence differing in 1 or 2 amino acids therefrom,
a heavy chain complementarity determining region 2 (CDRH2) having the amino acid sequence as shown in SEQ ID NO: 2, or an amino acid sequence differing in 1 or 2 amino acids therefrom,
a heavy chain complementarity determining region 3 (CDRH3) having the amino acid sequence as shown in SEQ ID NO: 3, or an amino acid sequence differing in 1 or 2 amino acids therefrom,
a light chain complementarity determining region 1 (CDRL1) having the amino acid sequence as shown in SEQ ID NO: 4, or an amino acid sequence differing in 1 or 2 amino acids therefrom,
a light chain complementarity determining region 2 (CDRL2) having the amino acid sequence as shown in SEQ ID NO: 5, or an amino acid sequence differing in 1 or 2 amino acids therefrom, and
a light chain complementarity determining region 3 (CDRL3) having the amino acid sequence as shown SEQ ID NOs: 6 or 7, or an amino acid sequence differing in 1 or 2 amino acids therefrom,
or an antibody recognizing an epitope within amino acids 342-652 of human HER2.

14. The method of claim 13, wherein the disease associated with HER2 overexpression, amplification and/or hyperactivity is a HER2 positive cancer or a metastasis of a HER2 positive cancer.

15. The method of claim 13, wherein the patient does not respond to monotherapy with the anti-HER2 antibody or to monotherapy with the second HER2 inhibitor.

16. The method of claim 13, wherein the patient does not respond to monotherapy with trastuzumab or pertuzumab.

17. The method of claim 13, wherein the disease associated with HER2 overexpression, amplification and/or hyperactivity is a HER2 positive cancer or a metastasis of a HER2 positive cancer.

18. The method of claim 17, wherein the cancer is lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasm of the central nervous system (CNS), spinal axis tumor, brain stem glioma, glioblastoma multiforme, astrocytoma, schwanoma, ependymona, medulloblastoma, meningioma, squamous cell carcinoma, pituitary adenoma, lymphoma, lymphocytic leukemia, including refractory versions of any of the above cancers, or a combination of one or more of the above cancers.

19. The method of claim 18, wherein the cancer is breast cancer, colon cancer, lung cancer, or pancreatic cancer.

20. The pharmaceutical composition or kit of claim 4, wherein the anti-HER2 antibody comprises a VH as shown in SEQ ID NO: 88 and a VL as shown in SEQ ID NO: 97.

21. The pharmaceutical composition or kit of claim 6, wherein the antibody does not bind an epitope between amino acids 342-652 of human HER2.

22. The pharmaceutical composition or kit of claim 8, wherein the one or more further cytotoxic, chemotherapeutic or anti-cancer agents comprises an alkylating agent, an anti-hormonal agent, an anti-proliferative agent, a radiopharmaceutical, or ionizing radiation.


SEQUENCE LISTING (97 sequences)

SEQ ID NO: 1 - protein, 5 aa, Artificial Sequence
  CDR-H1
NYGVS

SEQ ID NO: 2 - protein, 16 aa, Artificial Sequence
  CDR-H2
IISGSGFTYYASWAKG

SEQ ID NO: 3 - protein, 11 aa, Artificial Sequence
  CDR-H3
GVVPGYNAGGL

SEQ ID NO: 4 - protein, 11 aa, Artificial Sequence
  CDR-L1
QASQGISTALA

SEQ ID NO: 5 - protein, 7 aa, Artificial Sequence
  CDR-L2
SASTLAS

SEQ ID NO: 6 - protein, 12 aa, Artificial Sequence
  CDR-L3
QCTAAGSVSVGA

SEQ ID NO: 7 - protein, 12 aa, Artificial Sequence
  CDR-L3
QSTAAGSVSVGA

SEQ ID NO: 8 - protein, 29 aa, Artificial Sequence
  B100 FR-H1
QSVEESGGRLVTPGTPLTLTCTVSGFSLS

SEQ ID NO: 9 - protein, 30 aa, Artificial Sequence
  MAB237 FR-H1
QVQLEESGGRVVQPGTSLRLSCAASGFSLS

SEQ ID NO: 10 - protein, 30 aa, Artificial Sequence; identical to SEQ ID NO: 9
  MAB238 FR-H1

SEQ ID NO: 11 - protein, 30 aa, Artificial Sequence; identical to SEQ ID NO: 9
  MAB240 FR-H1

SEQ ID NO: 12 - protein, 30 aa, Artificial Sequence
  MAB241 FR-H1
EEHLEESGGRLVKPGTSLRLSCTVSGFSLS

SEQ ID NO: 13 - protein, 30 aa, Artificial Sequence; identical to SEQ ID NO: 9
  MAB267 FR-H1

SEQ ID NO: 14 - protein, 30 aa, Artificial Sequence; identical to SEQ ID NO: 9
  MAB268 FR-H1

SEQ ID NO: 15 - protein, 30 aa, Artificial Sequence; identical to SEQ ID NO: 9
  MAB269 FR-H1

SEQ ID NO: 16 - protein, 30 aa, Artificial Sequence; identical to SEQ ID NO: 12
  MAB270 FR-H1

SEQ ID NO: 17 - protein, 14 aa, Artificial Sequence
  B100 FR-H2
WVRQAPGKGLEYIG

SEQ ID NO: 18 - protein, 14 aa, Artificial Sequence
  MAB237 FR-H2
WVRQAPGKGLEYVA

SEQ ID NO: 19 - protein, 14 aa, Artificial Sequence
  MAB238 FR-H2
WVRQAPGKGLEYVA

SEQ ID NO: 20 - protein, 14 aa, Artificial Sequence
  MAB240 FR-H2
WVRQAPGKGLEYVA

SEQ ID NO: 21 - protein, 14 aa, Artificial Sequence
  MAB241 FR-H2
WVRQAPGRGLEYVS

SEQ ID NO: 22 - protein, 14 aa, Artificial Sequence
  MAB267 FR-H2
WVRQAPGKGLEYVA

SEQ ID NO: 23 - protein, 14 aa, Artificial Sequence
  MAB268 FR-H2
WVRQAPGKGLEYVA

SEQ ID NO: 24 - protein, 14 aa, Artificial Sequence
  MAB269 FR-H2
WVRQAPGKGLEYVA

SEQ ID NO: 25 - protein, 14 aa, Artificial Sequence
  MAB270 FR-H2
WVRQAPGRGLEYVS

SEQ ID NO: 26 - protein, 30 aa, Artificial Sequence
  B100 FR-H3
RFTISKTSTTVDLKITSPTTKDTATYFCAR

SEQ ID NO: 27 - protein, 32 aa, Artificial Sequence
  MAB237 FR-H3
RFTISKDTSKNTVVMQMTSLRAEDTATYFCAR

SEQ ID NO: 28 - protein, 32 aa, Artificial Sequence; identical to SEQ ID NO: 27
  MAB238 FR-H3

SEQ ID NO: 29 - protein, 32 aa, Artificial Sequence; identical to SEQ ID NO: 27
  MAB240 FR-H3

SEQ ID NO: 30 - protein, 32 aa, Artificial Sequence
  MAB241 FR-H3
RFTISKDTARDSVYLQMNSLRAEDTATYFCAR

SEQ ID NO: 31 - protein, 32 aa, Artificial Sequence; identical to SEQ ID NO: 27
  MAB267 FR-H3

SEQ ID NO: 32 - protein, 32 aa, Artificial Sequence; identical to SEQ ID NO: 27
  MAB268 FR-H3

SEQ ID NO: 33 - protein, 32 aa, Artificial Sequence; identical to SEQ ID NO: 27
  MAB269 FR-H3

SEQ ID NO: 34 - protein, 32 aa, Artificial Sequence; identical to SEQ ID NO: 30
  MAB270 FR-H3

SEQ ID NO: 35 - protein, 11 aa, Artificial Sequence
  B100 FR-H4
WGQGTLVTVSS

SEQ ID NO: 36 - protein, 11 aa, Artificial Sequence
  MAB237 FR-H4
WGQGTLVTVSS

SEQ ID NO: 37 - protein, 11 aa, Artificial Sequence
  MAB238 FR-H4
WGQGTLVTVSS

SEQ ID NO: 38 - protein, 11 aa, Artificial Sequence
  MAB240 FR-H4
WGQGTLVTVSS

SEQ ID NO: 39 - protein, 11 aa, Artificial Sequence
  MAB241 FR-H4
WGQGTLVTVSS

SEQ ID NO: 40 - protein, 11 aa, Artificial Sequence
  MAB267 FR-H4
WGQGTLVTVSS

SEQ ID NO: 41 - protein, 11 aa, Artificial Sequence
  MAB268 FR-H4
WGQGTLVTVSS

SEQ ID NO: 42 - protein, 11 aa, Artificial Sequence
  MAB269 FR-H4
WGQGTLVTVSS

SEQ ID NO: 43 - protein, 11 aa, Artificial Sequence
  MAB270 FR-H4
WGQGTLVTVSS

SEQ ID NO: 44 - protein, 23 aa, Artificial Sequence
  B100 FR-L1
DIVMTQTPASVSEPVGGTVTIKC

SEQ ID NO: 45 - protein, 23 aa, Artificial Sequence
  MAB237 FR-L1
DIQMTQSPSSLSASVGDRITITC

SEQ ID NO: 46 - protein, 23 aa, Artificial Sequence
  MAB238 FR-L1
DIVMTQSPSSVSASVGDRVTITC

SEQ ID NO: 47 - protein, 23 aa, Artificial Sequence
  MAB240 FR-L1
DIELTQSPSSVSASVGDRVTITC

SEQ ID NO: 48 - protein, 23 aa, Artificial Sequence
  MAB241 FR-L1
DIQMTQSPSSLSASVGDRITITC

SEQ ID NO: 49 - protein, 23 aa, Artificial Sequence
  MAB267 FR-L1
DIQMTQSPSSLSASVGDRITITC

SEQ ID NO: 50 - protein, 23 aa, Artificial Sequence
  MAB268 FR-L1
DIVMTQSPSSVSASVGDRVTITC

SEQ ID NO: 51 - protein, 23 aa, Artificial Sequence
  MAB269 FR-L1
DIELTQSPSSVSASVGDRVTITC

SEQ ID NO: 52 - protein, 23 aa, Artificial Sequence
  MAB270 FR-L1
DIQMTQSPSSLSASVGDRITITC

SEQ ID NO: 53 - protein, 15 aa, Artificial Sequence
  B100 FR-L2
WYQQKPGQRPKLLIY

SEQ ID NO: 54 - protein, 15 aa, Artificial Sequence
  MAB237 FR-L2
WYQQKPGQVPKLLIY

SEQ ID NO: 55 - protein, 15 aa, Artificial Sequence
  MAB238 FR-L2
WYQQKPGQAPKLLIY

SEQ ID NO: 56 - protein, 15 aa, Artificial Sequence
  MAB240 FR-L2
WYQQKPGQAPKLLIY

SEQ ID NO: 57 - protein, 15 aa, Artificial Sequence
  MAB241 FR-L2
WYQQKPGQVPKLLIY

SEQ ID NO: 58 - protein, 15 aa, Artificial Sequence
  MAB267 FR-L2
WYQQKPGQVPKLLIY

SEQ ID NO: 59 - protein, 15 aa, Artificial Sequence
  MAB268 FR-L2
WYQQKPGQAPKLLIY

SEQ ID NO: 60 - protein, 15 aa, Artificial Sequence
  MAB269 FR-L2
WYQQKPGQAPKLLIY

SEQ ID NO: 61 - protein, 15 aa, Artificial Sequence
  MAB270 FR-L2
WYQQKPGQVPKLLIY

SEQ ID NO: 62 - protein, 32 aa, Artificial Sequence
  B100 FR-L3
GVSSRFKGSGSGTQFTLTISDLECADAATYYC

SEQ ID NO: 63 - protein, 32 aa, Artificial Sequence
  MAB237 FR-L3
GVPSRFKGSGSGTEFTLTISSLQAEDVATYYC

SEQ ID NO: 64 - protein, 32 aa, Artificial Sequence
  MAB238 FR-L3
GVPSRFKGSGSGTDFTLTISSLQPEDSATYYC

SEQ ID NO: 65 - protein, 32 aa, Artificial Sequence
  MAB240 FR-L3
GVPSRFKGSGSGTDFTLTISSLQSEDSATYYC

SEQ ID NO: 66 - protein, 32 aa, Artificial Sequence; identical to SEQ ID NO: 63
  MAB241 FR-L3

SEQ ID NO: 67 - protein, 32 aa, Artificial Sequence; identical to SEQ ID NO: 63
  MAB267 FR-L3

SEQ ID NO: 68 - protein, 32 aa, Artificial Sequence; identical to SEQ ID NO: 64
  MAB268 FR-L3

SEQ ID NO: 69 - protein, 32 aa, Artificial Sequence; identical to SEQ ID NO: 65
  MAB269 FR-L3

SEQ ID NO: 70 - protein, 32 aa, Artificial Sequence; identical to SEQ ID NO: 63
  MAB270 FR-L3

SEQ ID NO: 71 - protein, 10 aa, Artificial Sequence
  B100 FR-L4
FGGGTEVVVN

SEQ ID NO: 72 - protein, 10 aa, Artificial Sequence
  MAB237 FR-L4
FGGGTEVVIK

SEQ ID NO: 73 - protein, 10 aa, Artificial Sequence
  MAB238 FR-L4
FGQGTELVIK

SEQ ID NO: 74 - protein, 10 aa, Artificial Sequence
  MAB240 FR-L4
FGGGTKVVIE

SEQ ID NO: 75 - protein, 10 aa, Artificial Sequence
  MAB241 FR-L4
FGGGTEVVIK

SEQ ID NO: 76 - protein, 10 aa, Artificial Sequence
  MAB267 FR-L4
FGGGTEVVIK

SEQ ID NO: 77 - protein, 10 aa, Artificial Sequence
  MAB268 FR-L4
FGQGTELVIK

SEQ ID NO: 78 - protein, 10 aa, Artificial Sequence
  MAB269 FR-L4
FGGGTKVVIE

SEQ ID NO: 79 - protein, 10 aa, Artificial Sequence
  MAB270 FR-L4
FGGGTEVVIK

SEQ ID NO: 80 - protein, 116 aa, Homo sapiens
QSVEESGGRLVTPGTPLTLTCTVSGFSLSNYGVSWVRQAPGKGLEYIGIISGSGFTYYASWAKGRFTISKTSTTVDLKITSPTTKDTATYFCARGVVPGYNAGGLWGQGTLVTVSS

SEQ ID NO: 81 - protein, 119 aa, Homo sapiens
QVQLEESGGRVVQPGTSLRLSCAASGFSLSNYGVSWVRQAPGKGLEYVAIISGSGFTYYASWAKGRFTISKDTSKNTVVMQMTSLRAEDTATYFCARGVVPGYNAGGLWGQGTLVTVSS

SEQ ID NO: 82 - protein, 119 aa, Homo sapiens; identical to SEQ ID NO: 81

SEQ ID NO: 83 - protein, 119 aa, Homo sapiens; identical to SEQ ID NO: 81

SEQ ID NO: 84 - protein, 119 aa, Homo sapiens
EEHLEESGGRLVKPGTSLRLSCTVSGFSLSNYGVSWVRQAPGRGLEYVSIISGSGFTYYASWAKGRFTISKDTARDSVYLQMNSLRAEDTATYFCARGVVPGYNAGGLWGQGTLVTVSS

SEQ ID NO: 85 - protein, 119 aa, Homo sapiens; identical to SEQ ID NO: 81

SEQ ID NO: 86 - protein, 119 aa, Homo sapiens; identical to SEQ ID NO: 81

SEQ ID NO: 87 - protein, 119 aa, Homo sapiens; identical to SEQ ID NO: 81

SEQ ID NO: 88 - protein, 119 aa, Homo sapiens; identical to SEQ ID NO: 84

SEQ ID NO: 89 - protein, 110 aa, Homo sapiens
DIVMTQTPASVSEPVGGTVTIKCQASQGISTALAWYQQKPGQRPKLLIYSASTLASGVSSRFKGSGSGTQFTLTISDLECADAATYYCQCTAAGSVSVGAFGGGTEVVVN

SEQ ID NO: 90 - protein, 110 aa, Homo sapiens
DIQMTQSPSSLSASVGDRITITCQASQGISTALAWYQQKPGQVPKLLIYSASTLASGVPSRFKGSGSGTEFTLTISSLQAEDVATYYCQCTAAGSVSVGAFGGGTEVVIK

SEQ ID NO: 91 - protein, 110 aa, Homo sapiens
DIVMTQSPSSVSASVGDRVTITCQASQGISTALAWYQQKPGQAPKLLIYSASTLASGVPSRFKGSGSGTDFTLTISSLQPEDSATYYCQCTAAGSVSVGAFGQGTELVIK

SEQ ID NO: 92 - protein, 110 aa, Homo sapiens
DIELTQSPSSVSASVGDRVTITCQASQGISTALAWYQQKPGQAPKLLIYSASTLASGVPSRFKGSGSGTDFTLTISSLQSEDSATYYCQCTAAGSVSVGAFGGGTKVVIE

SEQ ID NO: 93 - protein, 110 aa, Homo sapiens; identical to SEQ ID NO: 90

SEQ ID NO: 94 - protein, 110 aa, Homo sapiens
DIQMTQSPSSLSASVGDRITITCQASQGISTALAWYQQKPGQVPKLLIYSASTLASGVPSRFKGSGSGTEFTLTISSLQAEDVATYYCQSTAAGSVSVGAFGGGTEVVIK

SEQ ID NO: 95 - protein, 110 aa, Homo sapiens
DIVMTQSPSSVSASVGDRVTITCQASQGISTALAWYQQKPGQAPKLLIYSASTLASGVPSRFKGSGSGTDFTLTISSLQPEDSATYYCQSTAAGSVSVGAFGQGTELVIK

SEQ ID NO: 96 - protein, 110 aa, Homo sapiens
DIELTQSPSSVSASVGDRVTITCQASQGISTALAWYQQKPGQAPKLLIYSASTLASGVPSRFKGSGSGTDFTLTISSLQSEDSATYYCQSTAAGSVSVGAFGGGTKVVIE

SEQ ID NO: 97 - protein, 110 aa, Homo sapiens; identical to SEQ ID NO: 94